(19) United States
(12) Patent Application Publication
(10) Pub. No.: US 2026/0232486 A1
(43) Pub. Date: Aug. 13, 2026

(54) HEATED NASAL STRIP FOR HEATING A FACIAL REGION

(71) Applicant: Kimberly Bauman, Woodbury, MN (US)

(72) Inventor: Kimberly Bauman, Woodbury, MN (US)

(21) Appl. No.: 19/053,154

(22) Filed: Feb. 13, 2025

(51) Int. Cl. A61F 7/03; A61F 7/00

(52) U.S. Cl. CPC A61F 7/034; A61F 2007/0006

(57) ABSTRACT

A heated nasal strip for heating a facial region includes a body portion. The body portion is flat. The body portion is less than 8.0 inches in width and less than 3.0 inches in height. The body portion is air-permeable for flow of oxygen. An adhesive material is positioned on the body portion. The adhesive material is hypoallergenic for placement on skin. An oxygen-reactive material is positioned in the body portion. The oxygen-reactive material is designed to react to oxygen passing through the body portion to generate heat.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to nasal strips and more particularly pertains to a new heated nasal strip for heating a facial region. There are many articles of clothing and devices for keeping parts of the body warm. However, it can be difficult to keep facial regions warm without completely covering the facial region in a manner that is not aesthetically pleasing or that hinders talking and/or breathing. The new heated nasal strip can be adhered to the nose of a person to heat the person's nose without obstructing breathing or covering other areas of the facial region, such as the person's mouth.

### (2) Description of Related Art Including Information Disclosed Under 37 CFR 1.97 and 1.98

The prior art relates to nasal strips and nasal covers. The prior art, as best understood, does not disclose a heated nasal strip for heating a facial region that includes a body portion that is air-permeable for flow of oxygen, an adhesive material for placement on skin, and an oxygen-reactive material positioned in the body portion to react to oxygen passing through the body portion to generate heat.

### (h) BRIEF SUMMARY OF THE INVENTION

An embodiment of the disclosure meets the needs presented above in a heated nasal strip for heating a facial region generally comprising a body portion. The body portion is flat. The body portion is less than 8.0 inches in width and less than 3.0 inches in height. The body portion is air-permeable for flow of oxygen. An adhesive material is positioned on the body portion. The adhesive material is hypoallergenic for placement on skin. An oxygen-reactive material is positioned in the body portion. The oxygen-reactive material is designed to react to oxygen passing through the body portion to generate heat.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a heated nasal strip according to an embodiment of the disclosure.

FIG. 2 is a perspective view of an embodiment of the disclosure.

FIG. 3 is a perspective view of an embodiment of the disclosure in use.

FIG. 4 is a cross-sectional view of the embodiment of the disclosure in FIG. 1.

FIG. 5 is a front view of an embodiment of the disclosure in use.

FIG. 6 is a front view of an embodiment of the disclosure in use.

### (j) DETAILED DESCRIPTION OF THE INVENTION

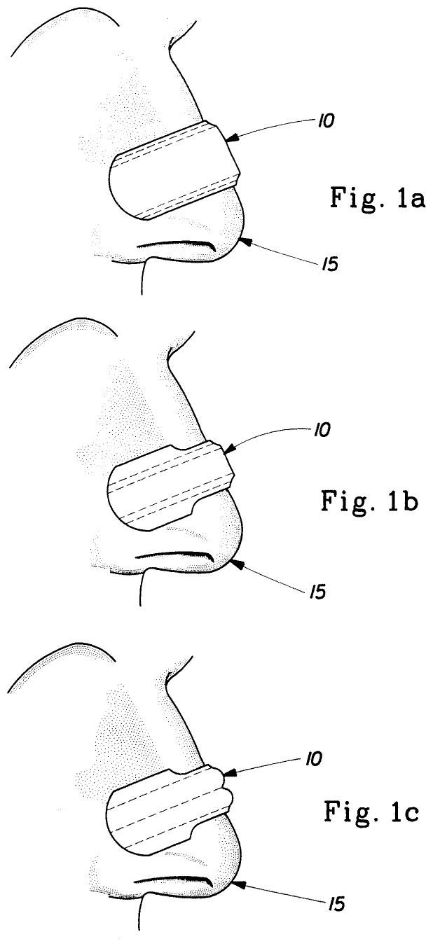

With reference now to the drawings, and in particular to FIGS. 1 through 6 thereof, a new heated nasal strip embodying the principles and concepts of an embodiment of the disclosure and generally designated by the reference numeral 10 will be described.

As best illustrated in FIGS. 1 through 6, the heated nasal strip 10 for heating a facial region 30 generally comprises a body portion 12. The body portion 12 is flat. The body portion 12 is less than 8.0 inches in width and less than 3.0 inches in height. The body portion 12 is air-permeable for flow of oxygen. An adhesive material 14 is positioned on the body portion 12. The adhesive material 14 is hypoallergenic for placement on skin. An oxygen-reactive material 16 is positioned in the body portion 12. The oxygen-reactive material 16 is designed to react to oxygen passing through the body portion 12 to generate heat.

The heated nasal strip 10 also includes a packet 18 enclosing the body portion 12. The packet 18 is openable for removal of the body portion 12 from the packet 18. The packet 18 is sealed to prevent exposure of the oxygen-reactive material 16 to oxygen prior to use. In the exemplary embodiment, the packet 18 is made of air-impermeable material.

The heated nasal strip 10 also includes a backing layer 20 that is removably adhered to the adhesive material 14 to cover the adhesive material 14 prior to use.

In the exemplary embodiment, the oxygen-reactive material 16 includes iron powder. The oxygen-reactive material 16 also includes activated carbon, vermiculite, and salt. In another possible embodiment, a different oxygen-reactive material 16 could be used to generate heat.

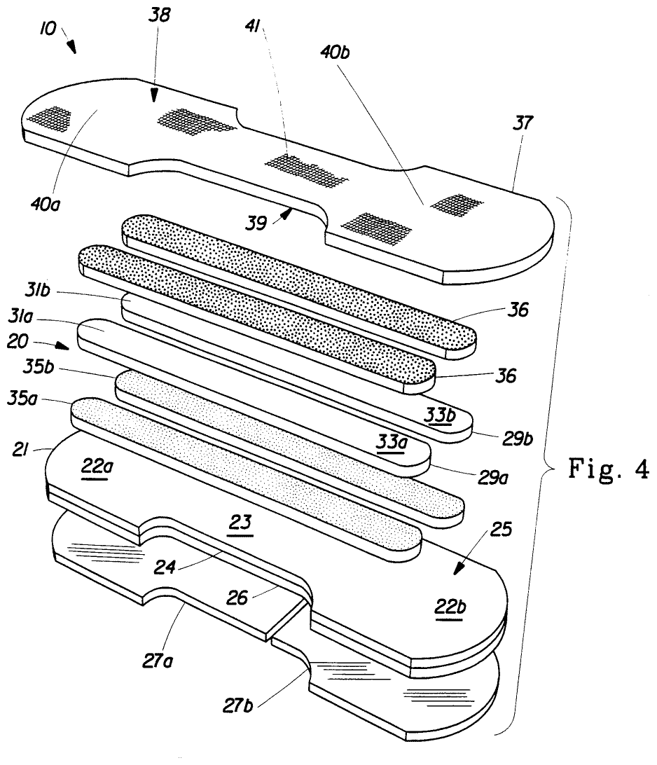

In the exemplary embodiment in FIG. 4, the body portion 12 includes a first layer 22. The adhesive material 14 is positioned on the first layer 22. The body portion 12 includes a second layer 24. The second layer 24 is made of an air-permeable fabric. The oxygen-reactive material 16 is positioned between the first layer 22 and the second layer 24. To further explain, the first layer 22 and the second layer 24 essentially form a pocket inside the body portion 12 that encloses the oxygen-reactive material 16. In one possible embodiment, the second layer 24 could also be made of air-permeable fabric.

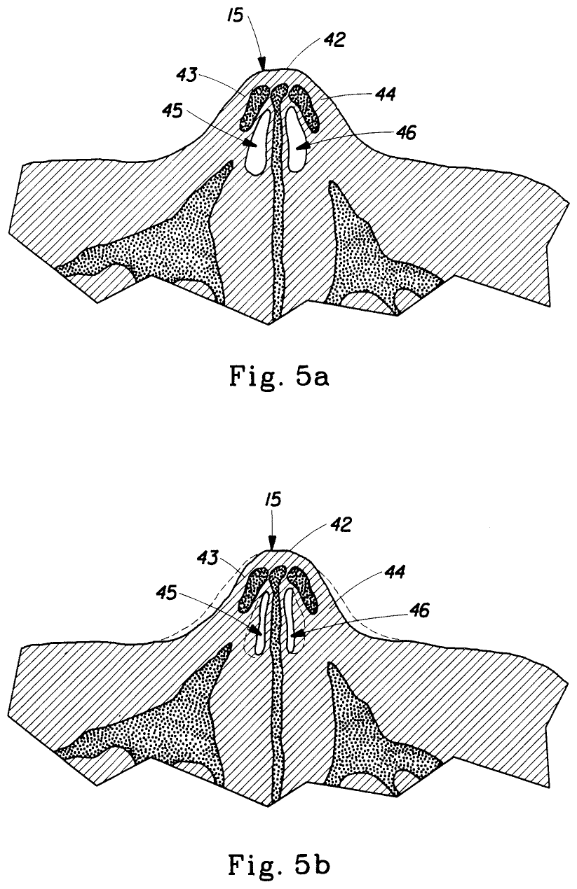

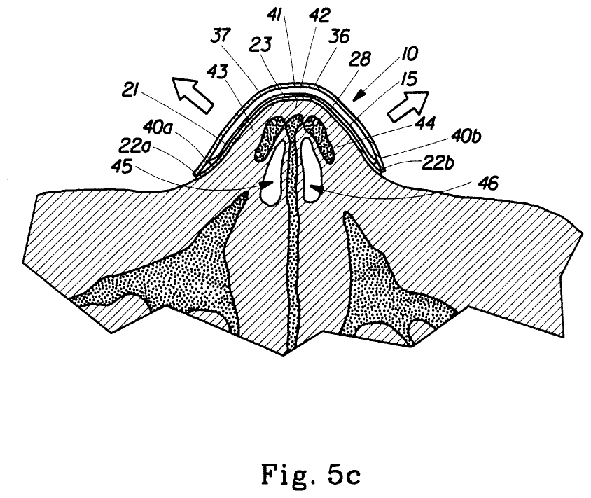

As shown in the exemplary embodiments in FIGS. 5 and 6, the body portion 12 is sized to fit on a face of a person over a nose 32. In the exemplary embodiment in FIG. 5, the body portion 12 is 1.5 to 2.5 inches in width and 0.75 to 1.5 inches in height. In the exemplary embodiment in FIG. 6, the body portion 12 is 4.0 to 6.0 inches in width and 0.75 to 1.5 inches in height. The body portion 12 is rectangular. However, in other possible embodiments, the body portion 12 is square, trapezoidal, oval-shaped, or butterfly-shaped.

In the exemplary embodiment, the second layer 24 has a skin-toned outer surface to generally match the skin tone of the person. In other possible embodiments, other colors could be used, or the second layer 24 could be transparent.

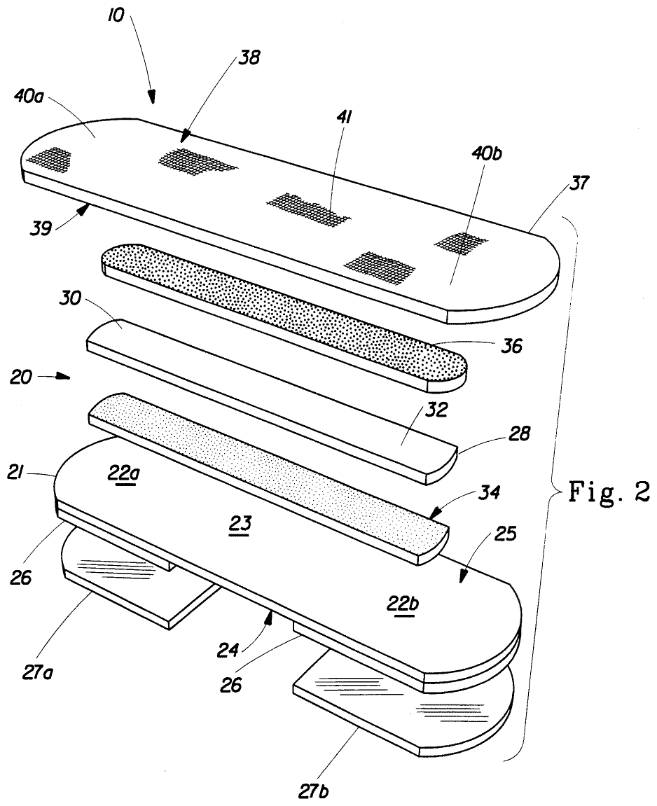

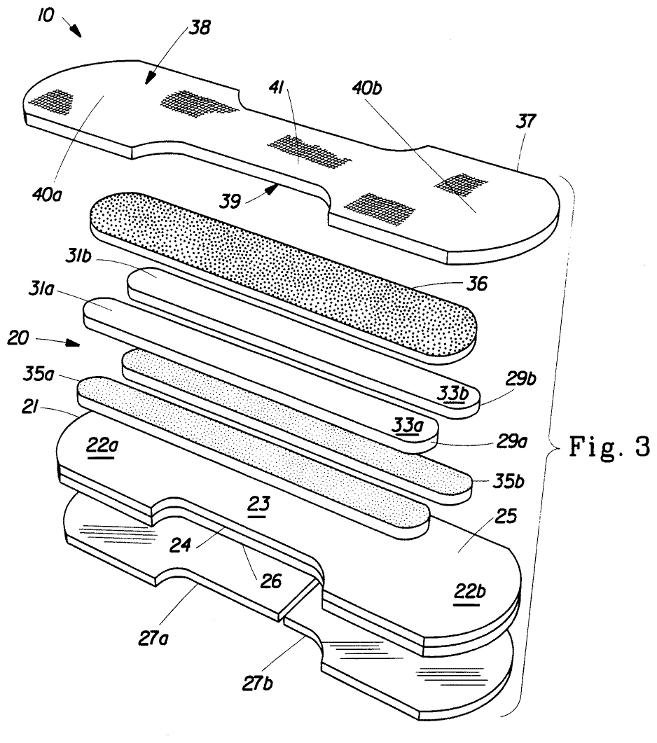

The heated nasal strip 10 is used to heat a facial region 30 of the person, such as the nose 32 in FIG. 5 or the nose 32 and cheeks 34 in FIG. 6. The person first removes the body portion 12 from the packet 18, as shown in FIG. 2, and then discards the packet 18. The person next removes the backing layer 20 to expose the adhesive material 14, as shown in FIG. 3. The person then places the body portion 12 across the nose 32 where desired and presses the adhesive material 14 against the skin. Air passes through the body portion 12 and reacts with the oxygen-reactive material 16. The chemical reaction generates heat to warm the nose 32 and any other portions of the facial region 30 in contact with or immediately adjacent the body portion 12. When done using the body portion 12, the person removes and discards the body portion 12.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heated nasal strip for heating a facial region comprising:
a body portion, said body portion being flat, said body portion being less than 8.0 inches in width and less than 3.0 inches in height, said body portion being air-permeable for flow of oxygen;
an adhesive material being positioned on said body portion, said adhesive material being hypoallergenic for placement on skin; and
an oxygen-reactive material being positioned in said body portion, said oxygen-reactive material being configured to react to oxygen passing through said body portion to generate heat.

2. The heated nasal strip of claim 1, further including a packet enclosing said body portion, said packet being openable for removal of said body portion from said packet, said packet being sealed to prevent exposure of said oxygen-reactive material to oxygen prior to use.

3. The heated nasal strip of claim 2, wherein said packet is made of air-impermeable material.

4. The heated nasal strip of claim 1, further including a backing layer being removably adhered to said adhesive material to cover said adhesive material prior to use.

5. The heated nasal strip of claim 1, wherein said oxygen-reactive material includes iron powder.

6. The heated nasal strip of claim 5, wherein said oxygen-reactive material includes activated carbon, vermiculite, and salt.

7. The heated nasal strip of claim 1, wherein:
said body portion includes a first layer;
said adhesive material is positioned on said first layer;
said body portion includes a second layer;
said second layer is made of an air-permeable fabric; and
said oxygen-reactive material is positioned between said first layer and said second layer.

8. The heated nasal strip of claim 1, wherein said body portion is 1.5 to 2.5 inches in width and 0.75 to 1.5 inches in height.

9. The heated nasal strip of claim 1, wherein said body portion is 4.0 to 6.0 inches in width and 0.75 to 1.5 inches in height.

10. The heated nasal strip of claim 1, wherein said body portion is rectangular.

11. The heated nasal strip of claim 1, wherein said second layer has a skin-toned outer surface.

12. The heated nasal strip of claim 3, further including a backing layer being removably adhered to said adhesive material to cover said adhesive material prior to use.

13. The heated nasal strip of claim 12, wherein said oxygen-reactive material includes iron powder.

14. The heated nasal strip of claim 13, wherein said oxygen-reactive material includes activated carbon, vermiculite, and salt.

15. The heated nasal strip of claim 14, wherein:
said body portion includes a first layer;
said adhesive material is positioned on said first layer;
said body portion includes a second layer;
said second layer is made of an air-permeable fabric; and
said oxygen-reactive material is positioned between said first layer and said second layer.

16. The heated nasal strip of claim 15, wherein:
said body portion is 1.5 to 2.5 inches in width and 0.75 to 1.5 inches in height;
said body portion is rectangular; and
said second layer has a skin-toned outer surface.

17. The heated nasal strip of claim 15, wherein:
said body portion is 4.0 to 6.0 inches in width and 0.75 to 1.5 inches in height;
said body portion is rectangular; and
said second layer has a skin-toned outer surface.
